(19) United States
(12) Patent Application Publication
(10) Pub. No.: US 2024/0337667 A1
(43) Pub. Date: Oct. 10, 2024

(54) METHOD FOR ESTIMATING THE PROBABILITY OF MALADAPTIVE GLOMERULAR IMPAIRMENT IN KIDNEY DISEASES

(71) Applicants: CENTRE HOSPITALIER UNIVERSITAIRE DE BORDEAUX, Talence (FR); ASSISTANCE PUBLIQUE HOPITAUX DE PARIS, Paris (FR); INSTITUT NATIONAL DE LA SANTÉ ET DE LA RECHERCHE MÉDICALE, Paris (FR); SORBONNE UNIVERSITE, Paris (FR)

(72) Inventors: Pierre GALICHON, Paris Cedex 20 (FR); Jean-Jacques BOFFA, Paris (FR); David BUOB, Paris (FR); Arthur ORIEUX, Bordeaux (FR); Charles VERNEY, Colombes (FR)

(21) Appl. No.: 18/713,251

(22) PCT Filed: Nov. 25, 2022
(86) PCT No.: PCT/EP2022/083348

§ 371 Date: May 24, 2024

(30) Foreign Application Priority Data
Nov. 26, 2021 (WO) PCT/IB2021/000835

(51) Int. Cl. G01N 33/68; A61K 31/573

(52) U.S. Cl. CPC G01N 33/6893; A61K 31/573; G01N 2333/765; G01N 2800/347

(57) ABSTRACT

An observational cohort with 40 patients (20 patients with primary FSGS and maladaptive FSGS, respectively) was carried out to identify renal morphometric parameters of interest. In addition, a validation cohort with 40 patients (20 patients with primary FSGS and maladaptive FSGS, respectively) was established to confirm the results matching age. estimated glomerular filtration rate (eGFR), and level of proteinuria. In the observational cohort, they found that the mean interglomerular area (MIA), a marker of glomerular scarcity described in the table 2) was significantly lower in patients with primary FSGS compared to maladaptive FSGS 90 [76-100] vs. 198 [165-299] μm2. p<0.0001. This finding was confirmed in the validation cohort 133 [109-159] vs. 204 [170-339] μm2 (p=0.0017). The present invention relates to a method for estimating the probability of maladaptive glomerular impairment in a subject comprising the following steps: i) obtaining a biological sample from said subject: ii) determining mean interglomerular area (MIA) value and iii) concluding that the subject has a high probability of maladaptive glomerular impairment when the MIA value is higher than the reference value: or concluding that the subject is not likely to have maladaptive glomerular impairment when the MIA value is lower than the reference value.

### FIELD OF THE INVENTION

The invention is in the kidney field. More particularly, the invention relates to a method for estimating the probability of maladaptive glomerular impairment in kidney diseases in a subject.

### BACKGROUND OF THE INVENTION

Glomerular hyperfiltration (GH) is a hallmark of renal dysfunction in diabetes and obesity. Glomerular hyperfiltration is associated with glomerular and tubular hypertrophy. Hyperfiltration is mainly due to an increase in glomerular capillary pressure, which increases tensile stress applied to the capillary wall structures. GH-related mechanical stress leads to both adaptive and maladaptive glomerular changes. Focal segmental glomerulosclerosis (FSGS) is both a glomerular lesion and renal disease. It refers to different diseases with very different pathophysiology and treatments¹. On the one hand, primary FSGSs is considered to be an immunological disease requiring immunosuppressive agents. On the other hand, maladaptive FSGS refers to the consequence of glomeruli hyperfiltration (especially in the context of nephron number reduction) requiring non-specific nephroprotective care. Pragmatically, the decision to treat with immunosuppressive agents (e.g., corticosteroid) is based on the probability that the FSGS is primary. However, it still involves a great deal of medical intuition. It mainly relies on unspecific and frequently unavailable information at the time of initial assessment: the speed of installation of the disease, the presence of a nephrotic syndrome (NS) with hypoalbuminemia <30 g/L, the percentage of podocyte foot process effacement assessed by electron microscopy (EM)⁴.

Thus, there is a need to find new tools to distinguish primary FSGS from maladaptive FSGS and avoid immunosuppressive treatment for the patients who have not to be treated with said treatment.

### SUMMARY OF THE INVENTION

The invention relates to a method for estimating the probability of maladaptive glomerular impairment in a subject comprising the following steps:
i) obtaining a biological sample from said subject;
ii) determining mean interglomerular area (MIA) value and
iii) concluding that the subject has a high probability of maladaptive glomerular impairment when the MIA value is higher than the reference value; or concluding that the subject is not likely to have maladaptive glomerular impairment when the MIA value is lower than the reference value.

In particular, the invention is defined by the claims.

### DETAILED DESCRIPTION OF THE INVENTION

Inventors hypothesize that the different pathophysiological mechanisms involved in immunosuppression-sensitive and maladaptive FSGS translate into different morphometric patterns that can be identified by optical microscopy, taking advantage of the recent availability of clinical-grade scanners allowing the large-scale digitalization of renal biopsies into high-definition whole slide images (WSI). An observational cohort with 40 patients (20 patients with primary FSGS and maladaptive FSGS, respectively) was carried out to identify renal morphometric parameters of interest. In addition, a validation cohort with 40 patients (20 patients with primary FSGS and maladaptive FSGS, respectively) was established to confirm the results matching age, estimated glomerular filtration rate (eGFR), and level of proteinuria.

In the observational cohort, they found that the mean interglomerular area (MIA), a marker of glomerular scarcity described in the Table 2 was significantly lower in patients with primary FSGS compared to maladaptive FSGS (90 [76-100] vs. 198 [165-299] μm², p<0.0001). This finding was confirmed in the validation cohort (133 [109-159] vs. 204 [170-339] μm2 (p=0.0017)).

MIA predictive performance measured by area under ROC (AUROC) curve was 0.96 (95% CI 0.89-1) and 0.83 (95% CI 0.66-1) for observation and validation cohort, respectively. Inventors determined an MIA cut-off value of 160,134 μm² to discriminate a primary FSGS from a maladaptive FSGS with a sensibility of 84% (95% CI 67-93) and a specificity of 85% (95% CI 68-94).

Accordingly, in a first aspect, the invention relates to a method for estimating the probability of maladaptive glomerular impairment in a subject comprising the following steps:
i) obtaining a biological sample from said subject;
ii) determining mean interglomerular area (MIA) and
iii) concluding that the subject has a high probability of maladaptive glomerular impairment when MIA is higher than the reference value; or concluding that the subject is not likely to have maladaptive glomerular impairment when MIA is lower than the reference value.

More particularly, the method according to the invention comprises further a step of associated MIA value with estimated glomerular filtration rate (eGFR), and/or level of proteinuria, albuminuria or albuminemia.

In a particular embodiment, the invention relates to a method for estimating the probability (p) of maladaptive glomerular impairment in a subject by comprising the following steps:
i) calculating the following formula:

p=1−1/(1+Exp(−5.69662497682848+0.00706798450932702×MIA−0.0345231327288216×eGFR+0.135805524527782×Albuminemia)); and
ii) concluding that the subject has a high probability of maladaptive glomerular impairment when the probability is higher than the reference value; or concluding that the subject not likely to have maladaptive glomerular impairment when the probability is lower than the reference value.

In a particular embodiment, the invention relates to a method for estimating the probability (p) of maladaptive glomerular impairment in a subject by comprising the following steps:
i) obtaining a biological sample from said subject;
ii) determining mean interglomerular area (MIA) value;
ii) associating MIA value determined at step ii) with estimated glomerular filtration rate (eGFR), level of albuminuria and albuminemia;
iii) calculating the probability of maladaptive glomerular impairment (p) by the following formula:

p=1−1/(1+Exp(−5.69662497682848+0.00706798450932702×MIA−0.0345231327288216×eGFR+0.135805524527782×Albuminemia)); and
iv) concluding that the subject has a high probability of maladaptive glomerular impairment when the probability is higher than the reference value; or concluding that the subject not likely to have maladaptive glomerular impairment when the probability is lower than the reference value.

More particularly, the method according to the invention comprises further a step of associated MIA value with age, glomerular filtration rate (eGFR), and/or level of proteinuria or albuminuria and albuminemia.

Accordingly, the invention relates to a method for estimating the probability (p) of maladaptive glomerular impairment in a subject comprising following steps:
i) obtaining a biological sample from said subject;
ii) determining mean interglomerular area (MIA) value;
iii) associating MIA value determined at step ii) with age, estimated glomerular filtration rate (eGFR), level of albuminuria and albuminemia;
iv) calculating the probability of maladaptive glomerular impairment (p) by the following formula:

p=1−1/(1+Exp ((−5.69662497682848)+(0.00625296091662126×MIA+0.0243941810080423×Age−0.0360256134329782×eGFR+0.161931298668144×Albuminemia+0.000255295983138449×Albuminuria)) and
v) concluding that the subject has a high probability of maladaptive glomerular impairment when the probability is higher than the reference value; or concluding that the subject not likely to have maladaptive glomerular impairment when the probability is lower than the reference value.

In a particular embodiment, the the invention relates to a method for estimating the probability (p) of maladaptive glomerular impairment in a subject comprising following steps:
i) obtaining a biological sample from said subject;
ii) determining mean interglomerular area (MIA) value;
iii) associating MIA value determined at step ii) with age, estimated glomerular filtration rate (eGFR), level of serum protein and proteinuria;
iv) calculating the probability of maladaptive glomerular impairment (p) by the following formula:

p=1−1/(1+Exp ((−5.69662497682848)+(0.00625296091662126×MIA+0.0243941810080423×Age−0.0360256134329782×eGFR+0.161931298668144×serum protein+0.000255295983138449×proteinuria)) and
v) concluding that the subject has a high probability of maladaptive glomerular impairment when the probability is higher than the reference value; or concluding that the subject not likely to have maladaptive glomerular impairment when the probability is lower than the reference value.

As used herein, the term “maladaptive glomerular impairment” refers to many diseases in kidney associated to the glomerular hyperfiltration (GH). Hyperfiltration is mainly due to an increase in glomerular capillary pressure, which increases tensile stress applied to the capillary wall structures. GH-related mechanical stress leads to both adaptive and maladaptive glomerular. Glomerular hyperfiltration is a characteristic functional abnormality in insulin-dependent diabetes mellitus and occurs in the large majority of young Type 1 diabetic patients. Hyperfiltration is hypothesized to be a precursor of intraglomerular hypertension leading to albuminuria. Glomerular filtration rate (GFR) then falls progressively in parallel with a further rise in albuminuria which may lead, in the long run, to end-stage renal failure. Hyperfiltration occurs in case of nephron number reduction, which can be caused by any kidney disease, and lead to maladaptive FSGS.

As used herein, the term “subject” refers to any mammals, such as a rodent, a feline, a canine, and a primate. Particularly, in the present invention, the subject is a human. In a particular embodiment, the subject is susceptible to suffer or is suffering from maladaptive glomerular impairment.

In a particular embodiment, the subject is susceptible to suffer or is suffering from diabetes mellitus.

As used herein, the term “diabetes mellitus” also known as diabetes, is a group of metabolic disorders characterized by a high blood sugar level over a prolonged period of time.

In a particular embodiment, the diabetes mellitus is type 1 diabetes winch results from failure of the pancreas to produce enough insulin due to loss of beta cells. This form is also called as “insulin-dependent diabetes mellitus”.

In a particular embodiment, the diabetes mellitus is type II diabetes mellitus (non-insulin-dependent diabetes mellitus or NIDDM). It is a metabolic disorder involving dysregulation of glucose metabolism and insulin resistance, and long-term complications involving the eyes, kidneys, nerves, and blood vessels. Type II diabetes mellitus usually develops in adulthood (middle life or later) and is described as the body's inability to make either sufficient insulin (abnormal insulin secretion) or its inability to effectively use insulin (resistance to insulin action in target organs and tissues)

In a further embodiment, the subject is susceptible to suffer or is suffering from a diabetic glomerulopathy.

As used herein, the term “diabetic glomerulopathy” refers to structural changes including glomerulosclerosis attributed to diabetes (in the context of uncontrolled diabetes and compatible associated features including but not restricted to nodular glomerulosclerosis, thickening of the basement membranes).

As used herein, the term “biological sample” refers to any sample obtained from a subject, such as a serum sample, a plasma sample, a urine sample, a blood sample, a lymph sample, or a biopsy. In a particular embodiment, the biological samples is a renal biopsy. Typically, biopsy sections were scanned (Pathscan Combi, Excilone), allowing to obtain high-definition whole slide images (WSI).

As used herein, the term “mean interglomerular area (MIA)” refers to a marker of glomerular scarcity. The MIA is calculated by dividing the minimum cortical area (minimum area between glomeruli, except GSG) by the number of glomeruli (except GSG) minus one (FIG. 1). MIA is a geometrical value depending on both the interglomerular distance and glomerular diameter.

In a particular embodiment, the method according to the invention wherein the mean interglomerular area (MIA) is calculated with the following formula=(minimal cortical area/total number of glomeruli except GSG−1)).

In the context of the invention, the total number of glomeruli (except GSG) is the total number of glomeruli found on the biological sample and must be ≥2, in a preferred embodiment, the total number of glomeruli (except GSG) is at least 10.

In a particular embodiment, the MIA value as determined above is incorporated in a software, a machine, R software (R Development Core Team, version 1.0.44) or GraphPad PRISM® Software (GraphPad Software, San Diego, USA, version 5.02, or ImageJ, Fiji, QuPath, TRIBVN Healthcare).

As used herein, the term “globally sclerotic glomeruli” (GSG) refers to terminally damaged glomeruli with no permeable capillary.

As used herein, the term “estimated glomerular filtration rate (eGFR)” also described as x3 refers to a measure of the renal function. This test measures the level of creatinine in the blood and uses the result in a formula to calculate a number that reflects how well the kidneys are functioning, said number is called eGFR. In the context of the invention, eGFR is calculated using the CKD-EPI formula with 4 categories, derived from the CKD classification (Chronic Kidney Disease):
i) category eGFR≥60 mL/min/1.73 m² is taken as a reference (condition with the lowest risk of the event)
ii) 3 other categories: 30-59 mL/min/1.73 m², 15-29 mL/min/1.73 m², <15 mL/min/1.73 m²

As used herein, the term “proteinuria” refers to the level of proteins in the urine. It is defined as a 24 h protein excretion or a protein/creatinine ratio. Typically, the level of proteinuria is determined in the urine sample by methods known in the art (in a biology lab).

As used herein, the term “serum protein” refers to the total amount of protein in the serum. The method well-known in the art for measuring total protein uses the biuret reagent, but other chemical methods such as Kjeldahl method, dye-binding and refractometry can also be used. The measurement is usually performed on automated analysers along with other laboratory tests.

As used herein, the term “albuminemia, also named herein “serum albumin concentration” refers to the level of albumin in the blood.

As used herein, the term “albuminuria” refers to the level of albumin in the urine. It is defined as a 24 h albumin excretion or an albumin/creatinine ratio. Typically, the level of albuminuria is determined in the urine sample by methods known in the art (in a biology lab)

In a second aspect, the invention relates to a method for discriminating primary Focal segmental glomerulosclerosis (FSGS) from maladaptive FSGS in a subject comprising determining mean interglomerular area (MIA) value from a biological sample obtained from said subject, preferably said method comprises the following steps:
i) obtaining a biological sample from said subject;
ii) determining mean interglomerular area (MIA) value and
iii) concluding that the subject is susceptible to suffer or is suffering from primary FSGS when MIA is lower than the reference value; or concluding that the subject is not susceptible to suffer or is not suffering from primary FSGS when MIA is higher than the reference valuc.

As used herein, the term “Focal segmental glomerulosclerosis (FSGS)” refers to a disease in which scar tissue develops on the parts of the kidneys that filter waste from the blood (glomeruli). FSGS is a serious condition that can lead to end-stage kidney disease, for which the only treatment options are dialysis or kidney transplant. Treatment options for FSGS depend on the type you have. There are different types of FSGS: i) primary FSGS: many people diagnosed with FSGS have no known cause for their condition. This is called primary (idiopathic) FSGS. Primary FSGS is considered to be an immunological disease requiring immunosuppressive agents; ii) maladaptive FSGS, also known as secondary FSGS refers to the consequence of glomeruli hyperfiltration requiring non-specific nephroprotective care. It is caused by various factors such as—but not limited to—infection, drug toxicity, diseases such as diabetes or sickle cell disease, obesity, small birth weight, congenital or acquired nephron number reduction and any other kidney disease.

The decision to treat with immunosuppressive agents (e.g., corticosteroid) is based on the probability that the FSGS is primary.

In another terms, the present disclosure relates to an in vitro method for discriminating primary Focal segmental glomerulosclerosis (FSGS) from maladaptive FSGS in a subject comprising determining mean interglomerular area (MIA) value in a biological sample, preferably renal biopsy sample, obtained from said subject, wherein said subject is susceptible to suffer or is suffering from primary FSGS when MIA is lower than a reference value; or the subject is susceptible to suffer or is suffering from maladaptive FSGS when MIA is higher than a reference value.

In a particular embodiment, the MIA is calculated with the following formula: MIA =(minimal cortical area/total number of glomeruli except segmental glomerulosclerosis (GSG)−1)).

The method for discriminating primary Focal segmental glomerulosclerosis (FSGS) from maladaptive FSGS as described above may further comprises the determination in said subject of at least one parameter selected from the group consisting of: estimated Glomerular Filtration Rate (eGFR), level of proteinuria such as albuminuria, level of serum protein such as serum albumin (also named albuminemia) and age, preferably estimated Glomerular Filtration Rate (eGFR), proteinuria and level of serum protein such as serum albumin.

In a preferred embodiment, the method according to the present disclosure further comprises determining the proteinuria in said subject and wherein the subject is susceptible to suffer or is suffering from primary FSGS when level of proteinuria (e.g. albuminuria) is higher than the reference value, or the subject is susceptible to suffer or is suffering from maladaptive FSGS when level of proteinuria (e.g. albuminuria) is lower than a reference value.

In another preferred embodiment, the method further comprises determining the level of serum protein, preferably serum albumin in a subject biological sample and wherein the subject is susceptible to suffer or is suffering from primary FSGS when said serum protein level is lower than a reference value, or the subject is susceptible to suffer or is suffering from maladaptive FSGS when serum protein level is higher than a reference value.

In another preferred embodiment, the method further comprises determining the estimated Glomerular Filtration Rate (eGFR) in a subject biological sample and wherein the subject is susceptible to suffer or is suffering from primary FSGS when eGFR is lower than a reference value, or the subject is susceptible to suffer or is suffering from maladaptive FSGS when eGFR is higher than a reference value.

In a particular embodiment, the invention relates to a method for discriminating primary Focal segmental glomerulosclerosis (FSGS) from maladaptive FSGS in a subject comprising the following steps:
i) calculating a combined value (p) with the following formula:

p=1−1/(1+Exp(−5.69662497682848+0.00706798450932702×MIA−0.0345231327288216×eGFR+0.135805524527782×Albuminemia)); and
ii) concluding that the subject is susceptible to suffer or is suffering from primary FSGS when the combined value is lower than the reference value; or concluding that the subject is not susceptible to suffer or is not suffering from primary FSGS when the combined value is higher than the reference value.

In a particular embodiment, the invention relates to a method for discriminating primary Focal segmental glomerulosclerosis (FSGS) from maladaptive FSGS in a subject comprising the following steps:
iii) obtaining a biological sample from said subject;
ii) determining mean interglomerular area (MIA) value; iii) associating MIA value with estimated glomerular filtration rate (eGFR), level of albuminuria by calculating a combined value (p) with the following formula:

p=1−1/(1+Exp(−5.69662497682848+0.00706798450932702×MIA−0.0345231327288216×eGFR+0.135805524527782×Albuminemia));
iv) concluding that the subject is susceptible to suffer or is suffering from primary FSGS when the combined value is lower than the reference value; or concluding that the subject is not susceptible to suffer or is not suffering from primary FSGS when the combined value is higher than the reference value.

In a further particular embodiment, the invention relates to a method for discriminating primary Focal segmental glomerulosclerosis (FSGS) from maladaptive FSGS in a subject comprising the following steps:
v) obtaining a biological sample from said subject;
ii) determining mean interglomerular area (MIA) value;
vi) associating MIA value with age, estimated glomerular filtration rate (eGFR), and level of albuminuria by calculating a combined value (p) with the following formula:

p=1−1/(1+Exp((−5.69662497682848)+(0.00625296091662126×MIA+0.0243941810080423×Age−0.0360256134329782×eGFR+0.161931298668144×Albuminemia+0.000255295983138449×Albuminuria))
iv) concluding that the subject is susceptible to suffer or is suffering from primary FSGS when the combined value with age, estimated glomerular filtration rate (eGFR), level of proteinuria and albuminuria is lower than the reference value; or concluding that the subject is not susceptible to suffer or is not suffering from primary FSGS when the combined value is higher than the reference value.

In a particular embodiment, the method of the invention further comprises the step of determining the proteinuria parameter.

In a preferred embodiment, the invention relates to a method for discriminating primary Focal segmental glomerulosclerosis (FSGS) from maladaptive FSGS in a subject comprising the following steps:
vii) obtaining a biological sample from said subject;
ii) determining mean interglomerular area (MIA) value;
viii) associating MIA value with age, estimated glomerular filtration rate (eGFR), and level of proteinuria by calculating a combined value (p) with the following formula:

p=1−1/(1+Exp((−5.69662497682848)+(0.00625296091662126×MIA+0.0243941810080423×Age−0.0360256134329782×eGFR+0.161931298668144×serum protein+0.000255295983138449×proteinuria))
iv) concluding that the subject is susceptible to suffer or is suffering from primary FSGS when the combined value with age, estimated glomerular filtration rate (eGFR), level of proteinuria and albuminuria is lower than the reference value; or concluding that the subject is not susceptible to suffer or is not suffering from primary FSGS when the combined value is higher than the reference value.

As used herein, the term “discriminating” refers to identify, observe a difference or distinguish two groups. Typically, the method according to the invention is suitable to identify or distinguish a subject who is susceptible to have primary or maladaptive FSGS.

As used herein, the term “subject” refers to any mammals, such as a rodent, a feline, a canine, and a primate. Particularly, in the present invention, the subject is a human, preferably susceptible to suffer or is suffering from FSGS. In a particular embodiment, the subject is susceptible to suffer or is suffering from primary FSGS. In a particular embodiment, the subject is susceptible to suffer or is suffering from maladaptive FSGS.

‘As used herein, the “reference value” refers to a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the MIA value in a group of reference, one can use algorithmic analysis for the statistic treatment of the value determined in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is high. This algorithmic method is preferably implemented by a computer executing code instructions stored on a memory. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In a preferred embodiment, the reference value of MIA to discriminate primary FSGS from maladaptive FSGC is between 100 μm² to 200 μm², preferably is between 120 μm² and 180 μm², more preferably between 140 μm² and 170 μm², again more preferably between 155 μm² and 165 μm². In another terms, a MIA value lower than 160 μm², preferably 140 μm², more preferably 120 μm², again more preferably 100 μm² is indicative that said subject is susceptible to suffer or is suffering from primary FSGS and a MIA value higher than 160 μm², preferably 170 μm², more preferably 180 μm² is indicative that said subject is susceptible to suffer or is suffering from primary FSGS and a MIA value higher.

In the context of the examples of the invention, the AUC value can be 0.96 for observational cohort. In a particular embodiment, the AUC value can be 0.83 for validation cohort. In a particular embodiment, the AUC value can be 0.88 for both cohorts. In a particular embodiment, the AUC value can be 0.91 for both cohorts, using a combination of MIA value minus age, estimated glomerular filtration rate (eGFR), level of serum protein and level of urine protein.

In the context of the invention, the analyses can be performed with R software (R Development Core Team, version 1.0.44) and GraphPad PRISM® Software (GraphPad Software, San Diego, USA, version 5.02).

The distinction between primary and maladaptive FSGS is not always obvious, resulting in several patients with maladaptive FSGS undergoing unnecessary and potential harmful immunosuppressive therapy. Therefore, inventors propose to use MIA, an easily assessable morphometric parameter at the time of renal biopsy, to predict corticosteroid sensitivity.

Accordingly, in a third aspect, the invention relates to a method for predicting whether a subject suffering from maladaptive glomerular impairment in kidneys will achieve a response to an immunosuppressive treatment comprising the following steps:
i) Obtaining a biological sample from said subject;
ii) determining mean interglomerular area (MIA) and
ii) concluding that the subject will achieve a response to an immunosuppressive treatment when MIA is lower than the reference value; or concluding that the subject will not achieve a response to an immunosuppressive treatment when MIA is higher than the reference value.

More particularly, the method according to the invention comprises further a step of associated MIA value with glomerular filtration rate (eGFR), and/or level of proteinuria or albuminuria and albuminemia.

In a particular embodiment, the invention relates to a method for predicting whether a subject suffering from maladaptive glomerular impairment in kidneys will achieve a response to an immunosuppressive treatment comprising the following steps:
i) calculating a combined value (p) with the following formula:

p=1−1/(1+Exp(−5.69662497682848+0.00706798450932702×MIA−0.0345231327288216×eGFR+0.135805524527782×Albuminemia)); and
ii) concluding that the subject will achieve a response to an immunosuppressive treatment when the combined value is lower than the reference value; or concluding that the subject will not achieve a response to an immunosuppressive treatment when the combined value is higher than the reference value.

In a further particular embodiment, the method according to the invention comprises further a step of associated MIA value with age.

In a particular embodiment, the invention relates to a method for predicting whether a subject suffering from maladaptive glomerular impairment in kidneys will achieve a response to an immunosuppressive treatment comprising the following steps:
i) calculating a combined value (p) with the following formula:

p=1−1/(1+Exp((−5.69662497682848)+(0.00625296091662126*MIA+0.0243941810080423×Age−0.0360256134329782×eGFR+0.161931298668144×Albuminemia+0.000255295983138449×Albuminuria)), and
ii) concluding that the subject will achieve a response to an immunosuppressive treatment when the combined value is lower than the reference value; or concluding that the subject will not achieve a response to an immunosuppressive treatment when the combined value is higher than the reference value.

In a further embodiment, the method according to the invention comprises further a step of associated MIA value with proteinuria.

In a particular embodiment, the invention relates to a method for predicting whether a subject suffering from maladaptive glomerular impairment in kidneys will achieve a response to an immunosuppressive treatment comprising the following steps:
i) calculating a combined value (p) with the following formula:

p=1−1/(1+Exp((−5.69662497682848)+(0.00625296091662126×MIA+0.0243941810080423×Age−0.0360256134329782×eGFR+0.161931298668144×serum protein+0.000255295983138449×proteinuria)), and
ii) concluding that the subject will achieve a response to an immunosuppressive treatment when the combined value is lower than the reference value; or concluding that the subject will not achieve a response to an immunosuppressive treatment when the combined value is higher than the reference value.

In a particular embodiment, the invention relates to a method for predicting whether a subject suffering from primary or maladaptive FSGS will achieve a response to an immunosuppressive treatment comprising the following steps:
i) Obtaining a biological sample from said subject;
ii) determining mean interglomerular area (MIA) and
iii) concluding that the subject will achieve a response to an immunosuppressive treatment when MIA is lower than the reference value; or concluding that the subject will not achieve a response to an immunosuppressive treatment when MIA is higher than the reference value.

In a particular embodiment, the invention relates to a method for predicting whether a subject suffering from primary or maladaptive FSGS will achieve a response to an immunosuppressive treatment comprising the following steps:
i) calculating a combined value (p) with the following formula:

p=1−1/(1+Exp(−5.69662497682848+0.00706798450932702×MIA−0.0345231327288216×eGFR+0.135805524527782×Albuminemia)); and
ii) concluding that the subject will achieve a response to an immunosuppressive treatment when the combined value is lower than the reference value; or concluding that the subject will not achieve a response to an immunosuppressive treatment when the combined value is higher than the reference value.

In a particular embodiment, the invention relates to a method for predicting whether a subject suffering from primary or maladaptive FSGS will achieve a response to an immunosuppressive treatment comprising the following steps:
i) calculating a combined value (p) with the following formula:

p=1−1/(1+Exp((−5.69662497682848)+(0.00625296091662126×MIA+0.0243941810080423×Age−0.0360256134329782×eGFR+0.161931298668144×Albuminemia+0.000255295983138449×Albuminuria))
ii) concluding that the subject will achieve a response to an immunosuppressive treatment when the combined value is lower than the reference value; or concluding that the subject will not achieve a response to an immunosuppressive treatment when the combined value is higher than the reference value.

In a particular embodiment, the invention relates to a method for predicting whether a subject suffering from primary or maladaptive FSGS will achieve a response to an immunosuppressive treatment comprising the following steps:
i) calculating a combined value (p) with the following formula:

p=1−1/(1+Exp((−5.69662497682848)+(0.00625296091662126×MIA+0.0243941810080423×Age−0.0360256134329782×eGFR+0.161931298668144×serum protein+0.000255295983138449×proteinuria)), and
ii) concluding that the subject will achieve a response to an immunosuppressive treatment when the combined value is lower than the reference value; or concluding that the subject will not achieve a response to an immunosuppressive treatment when the combined value is higher than the reference value.

In a particular embodiment, the invention relates to a method for predicting the probability of response to an immunosuppressive treatment in a subject suffering from maladaptive glomerular impairment in kidneys comprising the following steps:
i) Obtaining a biological sample from said subject;
ii) determining mean interglomerular area (MIA) and
iii) concluding that the probability that the subject will respond to an immunosuppressive treatment when MIA is lower than the reference value; or concluding that the subject will likely not respond to an immunosuppressive treatment when MIA is higher than the reference value.

In a particular embodiment, the invention relates to a method for predicting the probability of response to an immunosuppressive treatment in a subject suffering from maladaptive glomerular impairment in kidneys comprising the following steps:
i) calculating a combined value (p) with the following formula:

p=1−1/(1+Exp(−5.69662497682848+0.00706798450932702×MIA−0.0345231327288216×eGFR+0.135805524527782×Albuminemia)); and
ii) concluding that the probability that the subject will respond to an immunosuppressive treatment when the combined value is lower than the reference value; or concluding that the subject will likely not respond to an immunosuppressive treatment when the combined value is higher than the reference value.

In a particular embodiment, the invention relates to a method for predicting the probability of response to an immunosuppressive treatment in a subject suffering from maladaptive glomerular impairment in kidneys comprising the following steps:
i) calculating a combined value (p) with the following formula:

p=1−1/(1+Exp((−5.69662497682848)+(0.00625296091662126×MIA+0.0243941810080423×Age−0.0360256134329782×eGFR+0.161931298668144×Albuminemia+0.000255295983138449×Albuminuria))
ii) concluding that the probability that the subject will respond to an immunosuppressive treatment when the combined value is lower than the reference value; or concluding that the subject will likely not respond to an immunosuppressive treatment when the combined value is higher than the reference value.

In a particular embodiment, the invention relates to a method for predicting the probability of response to an immunosuppressive treatment in a subject suffering from maladaptive glomerular impairment in kidneys comprising the following steps:
i) calculating a combined value (p) with the following formula:

p=1−1/(1+Exp((−5.69662497682848)+(0.00625296091662126×MIA+0.0243941810080423×Age−0.0360256134329782×eGFR+0.161931298668144×serum protein+0.000255295983138449×proteinuria)),and
ii) concluding that the probability that the subject will respond to an immunosuppressive treatment when the combined value is lower than the reference value; or concluding that the subject will likely not respond to an immunosuppressive treatment when the combined value is higher than the reference value.

In a particular embodiment, the invention relates to a method for predicting the probability of response to an immunosuppressive treatment in a subject suffering from primary or maladaptive FSGS comprising the following steps:
i) Obtaining a biological sample from said subject;
ii) determining mean interglomerular area (MIA) and
iii) concluding that the probability that the subject will respond to an immunosuppressive treatment when MIA is lower than the reference value; or concluding that the subject will likely not respond to an immunosuppressive treatment when MIA is higher than the reference value.

In a particular embodiment, the invention relates to a method for predicting the probability of response to an immunosuppressive treatment in a subject suffering from primary or maladaptive FSGS comprising the following steps:
i) calculating a combined value (p) with the following formula:

p=1−1/(1+Exp(−5.69662497682848+0.00706798450932702×MIA−0.0345231327288216×eGFR+0.135805524527782×Albuminemia)); and
ii) concluding that the probability that the subject will respond to an immunosuppressive treatment when the combined value is lower than the reference value; or concluding that the subject will likely not respond to an immunosuppressive treatment when the combined value with is higher than the reference value.

In a particular embodiment, the invention relates to a method for predicting the probability of response to an immunosuppressive treatment in a subject suffering from primary or maladaptive FSGS comprising the following steps:
i) calculating a combined value (p) with the following formula:

p=1−1/(1+Exp((−5.69662497682848)+(0.00625296091662126×MIA+0.0243941810080423×Age−0.0360256134329782×eGFR+0.161931298668144×Albuminemia+0.000255295983138449×Albuminuria)) and
ii) concluding that the probability that the subject will respond to an immunosuppressive treatment when the combined value is lower than the reference value; or concluding that the subject will likely not respond to an immunosuppressive treatment when the combined value with is higher than the reference value.

In a particular embodiment, the invention relates to a method for predicting the probability of response to an immunosuppressive treatment in a subject suffering from primary or maladaptive FSGS comprising the following steps:
i) calculating a combined value (p) with the following formula:

p=1−1/(1+Exp((−5.69662497682848)+(0.00625296091662126×MIA+0.0243941810080423×Age−0.0360256134329782×eGFR+0.161931298668144×serum protein+0.000255295983138449×proteinuria)),
ii) concluding that the probability that the subject will respond to an immunosuppressive treatment when the combined value is lower than the reference value; or concluding that the subject will likely not respond to an immunosuppressive treatment when the combined value with is higher than the reference value.

In another particular embodiment, the invention relates to an in vitro method for predicting whether a subject suffering from FSGS will respond to an immunosuppressive treatment comprising determining mean interglomerular area (MIA) value in a biological sample obtained from said subject, wherein said subject will respond to an immunosuppressive treatment when MIA is lower than a reference value; or the subject will not respond to an immunosuppressive treatment when MIA is higher than a reference value. In a preferred embodiment, the MIA is calculated with the following formula: MIA=(minimal cortical area/total number of glomeruli except segmental glomerulosclerosis (GSG)−1)).

The method for predicting whether a subject suffering from FSGS will respond to an immunosuppressive treatment as described above may further comprise the determination in said subject of at least one parameter selected from the group consisting of: estimated Glomerular Filtration Rate (eGFR), level of proteinuria (e.g. albuminuria), level of serum protein such as serum albumin and age, preferably estimated Glomerular Filtration Rate (eGFR), level of proteinuria (e.g. albuminuria) and level of serum protein such as serum albumin.

In a preferred embodiment, the method according to the present disclosure further comprises determining the level of proteinuria (e.g. albuminuria) in said subject and wherein the subject is susceptible to respond to an immunosuppressive treatment when the level of proteinuria is higher than the reference value, or the subject is not susceptible to respond an immunosuppressive treatment when the level of proteinuria (e.g. albuminuria) is lower than a reference value.

In another preferred embodiment, the method further comprises determining the level of serum protein, preferably serum albumin in a subject biological sample and wherein the subject is susceptible to respond to an immunosuppressive treatment when said serum protein level is lower than a reference value, or the subject is not susceptible to respond to an immunosuppressive treatment when serum protein level concentration is higher than a reference value.

In another preferred embodiment, the method further comprises determining the estimated Glomerular Filtration Rate (eGFR) in a subject biological sample and wherein the subject is susceptible to respond to an immunosuppressive treatment when eGFR is lower than a reference value, or the subject is not susceptible to respond to an immunosuppressive treatment when eGFR is higher than a reference value.

As used herein, the term “predicting” means that the subject to be analyzed by the method of the invention is allocated either into the group of subjects who will relapse, or into a group of subjects who will not relapse after a treatment.

As used herein, the term “risk” in the context of the present invention, relates to the probability that an event will occur over a specific time period, as in the conversion to relapse, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion. “Risk evaluation,” or “evaluation of risk” in the context of the present invention encompasses making a prediction of the probability, odds, or likelihood that an event or disease state may occur, the rate of occurrence of the event or conversion from one disease state to another, i.e., from a normal condition to relapse or to one at risk of developing relapse. Risk evaluation can also comprise prediction of future clinical parameters, traditional laboratory risk factor values, or other indices of relapse, either in absolute or relative terms in reference to a previously measured population. The methods of the present invention may be used to make continuous or categorical measurements of the risk of conversion to relapse, thus diagnosing and defining the risk spectrum of a category of subjects defined as being at risk of having relapse. In the categorical scenario, the invention can be used to discriminate between normal and other subject cohorts at higher risk of having relapse. In some embodiments, the present invention may be used so as to discriminate those at risk of having relapse from normal, or those having relapse disease from normal.

As used herein, the terms “will achieve a response” or “respond” refer to the response to a treatment of the subject suffering from primary or maladaptive FSGS. Typically, such treatment induces, ameliorates or otherwise causes an improvement in the pathological symptoms, disease progression or physiological conditions associated with or resistance to succumbing to a maladaptive FSGS. In particular, in the context of the invention, the term “respond” refers to the ability of corticosteroid treatment to an improvement of the pathological symptoms, thus, the subject presents a clinical improvement compared to the subject who does not receive the treatment. The said subject is considered as a “responder” to the treatment. The term “not respond” refers to a subject who does not present any clinical improvement to the treatment with a corticosteroid treatment. This subject is considered as a “non-responder” to the treatment. Accordingly, the subject as considered “non-responder” has a particular monitoring in the therapeutic regimen. In a particular embodiment, the response to a treatment is determined by an increase of proteinemia level (e.g., albuminemia), a decrease of proteinuria level (e.g., albuminuria), an improvement of renal function that can be characterized by estimated Glomerular Filtration Rate (cGFR) and/or a reduction of FSGS lesion in biopsy. These criteria refer to a set of published rules that define when the disease in the subjects improve (“respond”), stay the same (“stabilize”), or worsen (“progress”) during treatment. In the context of the invention, when the subject is identified as responder, it means that said subject improves one or more of these criteria. More particularly, the MIA value is a tool to determine the overall survival (OS) of the subject at the time of renal biopsy to an immunosuppressive treatment.

As used herein, the term “relapse” refers to the return of signs and symptoms of a disease after a subject has enjoyed a remission after a treatment. Thus, if initially the target disease is alleviated or healed, or progression of the disease was halted or slowed down, and subsequently the disease or one or more characteristics of the disease return, the subject is referred to as being “relapsed.”

In a particular embodiment, the biological sample as described above is a renal biopsy. Typically, biopsy sections were scanned (Pathscan Combi, Excilone), allowing to obtain high-definition whole slide images (WSI).

In a particular embodiment, the mean interglomerular area (MIA) as described above is calculated with the following formula=(minimal cortical area/total number of glomeruli except GSG−1)). In a preferred embodiment, the MIA is measured on a total number of at least 10 glomeruli except GSC.

According to a particular embodiment, the total number of glomeruli (except GSG) may be equal to 14. According to a particular embodiment, the minimal cortical area may be 1052838.56 μm².

In a particular embodiment, the MIA value as determined above is incorporated in a soft ware, a machine, R software (R Development Core Team, version 1.0.44) or GraphPad PRISM® Software (GraphPad Software, San Diego, USA, version 5.02, ImageJ, Fiji, QuPath, TRIBVN Healthcare).

In a particular embodiment, the method according to the invention is performed at the time of renal biopsy.

In a fourth aspect, the present disclosure relates to an immunosuppressive agent for use in the treatment of a Focal segmental glomerulosclerosis in a subject in need thereof, wherein immunosuppressive drug is administered in a subject previously identified as susceptible to suffer or is suffering from primary FSGS or to respond to immunosuppressive treatment according to any one of the methods described above.

The present disclosure also relates to a method for treating a Focal segmental glomerulosclerosis in a subject in need thereof previously identified as susceptible to suffer or is suffering from primary FSGS or to respond to immunosuppressive treatment according to any one of the methods described above, said method comprising administering a therapeutically effective amount of an immunosuppressive agent in said subject.

In the context of the disclosure, the term “treating” or “treatment”, as used herein, means reversing, alleviating or inhibiting the progress of the disease caused by FSGS, preferably primary FSGS or reversing, alleviating or inhibiting the progress of one or more symptoms of the disorder or condition to which such term applies.

By “therapeutically effective amount” refers to an amount effective, at dosages and for periods of time necessary to achieve the desired therapeutic result. The therapeutically effective amount of the product of the disclosure that comprises it may vary according to factors such as the disease state, age, sex, and weight of the individual, and the ability of the product or pharmaceutical composition to elicit a desired response in the individual. Dosage regimens may be adjusted to provide the optimum therapeutic response. A therapeutically effective amount is also typically one in which any toxic or detrimental effect of the product or pharmaceutical composition is outweighed by the therapeutically beneficial effects.

As used herein, the term “subject” refers to any mammals, such as a rodent, a feline, a canine, and a primate. Particularly, in the present invention, the subject is a human. In a particular embodiment, the subject is susceptible to suffer or is suffering from FSGS, preferably primary FSGS.

As used herein, the term “immunosuppressive treatment” refers to a treatment that suppresses some immune function. Immunosuppressive treatment means that the subject is administered with one or more immunosuppressive drugs. Immunosuppressive drugs or other drugs that are currently known in the art or that will be identified in the future. In a particular embodiment, the immunosuppressive drugs include but not limited to azathioprine, tacrolimus, rapamycin derivative (sirolimus and everolimus), mycophenolic acid (mycophenolate mofetil and enteric-coated mycophenolate sodium), corticosteroids, and cyclosporin, anti CD20 antibodies (rituximab, obinutuzumab), cyclophosphamide, chlorambucil. These drugs may be used in monotherapy or in combination therapies.

In a particular embodiment, the immunosuppressive treatment is corticosteroid treatment. As used, the term “corticosteroid treatment” has its general meaning in the art and refers to a treatment with a class of active ingredients having a hydrogenated cyclopentoperhydrophenanthrene ring system endowed with an anti-inflammatory activity. Corticosteroid drugs typically include cortisone, cortisol, hydrocortisone (11β,17-dihydroxy, 21-(phosphonooxy)-pregn-4-ene, 3,20-dione disodium), dihydroxycortisone, dexamethasone (21-(acetyloxy)-9-fluoro-1β,17-dihydroxy-16α-m-ethylpregna-1,4-diene-3,20-dione), and highly derivatized steroid drugs such as beconase (beclomethasone dipropionate, which is 9-chloro-11-β, 17,21, trihydroxy-16β-methylpregna-1,4 diene-3,20-dione 17,21-dipropionate). Other examples of corticosteroids include flunisolide, prednisone, prednisolone, methylprednisolone, triamcinolone, deflazacort and betamethasone. corticosteroids, for example, cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinolone, fluocinonide, betamethasone dipropionate, betamethasone valerate, desonide, desoximetasone, fluocinolone, triamcinolone, triamcinolone acetonide, clobetasol propionate, and dexamethasone, and endogenous glucocorticoid stimulating agents (adrenocorticotropic hormone).

The methods as described above are implemented by a computer executing code instructions stored on a memory.

Accordingly, in a fifth aspect, the invention relates to a computer-implemented method for performing any one of the method as described above, preferably an in vitro method for discriminating primary Focal segmental glomerulosclerosis (FSGS) from maladaptive FSGS in a subject comprising determining mean interglomerular area (MIA) value in a biological sample obtained from said subject, wherein said subject is susceptible to suffer or is suffering from primary FSGS when MIA is lower than a reference value; or the subject is susceptible to suffer or is suffering from maladaptive FSGS when MIA is higher than a reference value.

In another embodiment, the invention relates to a computer-implemented method for predicting whether a subject suffering from FSGS will respond to an immunosuppressive treatment comprising determining mean interglomerular area (MIA) value in a biological sample, preferably a renal biopsy sample, obtained from said subject, wherein said subject will respond to an immunosuppressive treatment when MIA is lower than a reference value; or the subject will not respond to an immunosuppressive treatment when MIA is higher than a reference value.

In a particular embodiment, the present invention relates to the computer-implemented method as described above comprising the following steps:
i) incorporating in a software a set of parameters comprising MIA value determined in a biological sample obtained from a subject, and preferably at least one of the parameters selected from the group consisting of: estimated glomerular filtration rate (eGFR), level of proteinuria (e.g., albuminuria) and serum protein level such as serum albumin level in said subject,
ii) calculating a probability of maladaptive FSGS (p) from said set of parameters, and
iii) concluding that said subject is susceptible to suffer or is suffering from primary FSGS or to respond to immunosuppressive treatment when said probability is lower than a reference value; or the subject is susceptible to suffer or is suffering from maladaptive FSGS or not to respond to immunosuppressive treatment when said probability is higher than a reference value.

Typically, the invention relates to a computer-implemented method for estimating the probability of maladaptive glomerular impairment, discriminating primary Focal segmental glomerulosclerosis (FSGS) from maladaptive FSGS and/or for predicting the probability of response to an immunosuppressive treatment in a subject.

In a particular embodiment, the invention relates to a computer-implemented method for estimating the probability of maladaptive glomerular impairment in a subject comprising the following steps:
i) obtaining a biological sample from said subject;
ii) determining mean interglomerular area (MIA);
iii) incorporating the MIA value determined at step ii) in a software; and
iv) concluding that the subject has a high probability of maladaptive glomerular impairment when MIA is higher than the reference value; or concluding that the subject is not likely to have maladaptive glomerular impairment when MIA is lower than the reference value.

In a further embodiment, the invention relates to a computer-implemented method for estimating the probability of maladaptive glomerular impairment in a subject comprising the following steps:
i) calculating a combined value (p) with the following formula:

p=1−1/(1+Exp(−5.69662497682848+0.00706798450932702×MIA−0.0345231327288216×eGFR+0.135805524527782×Albuminemia));
ii) incorporating the combined value in a software; and
iii) concluding that the subject has a high probability of maladaptive glomerular impairment when the combined value is higher than the reference value; or concluding that the subject not likely to have maladaptive glomerular impairment when the combined value is lower than the reference value.

In a further embodiment, the invention relates to a computer-implemented method for estimating the probability of maladaptive glomerular impairment in a subject comprising the following steps:
i) calculating a combined value (p) with the following formula:

p=1−1/(1+Exp((−5.69662497682848)+(0.00625296091662126*MIA+0.0243941810080423×Age−0.0360256134329782×eGFR+0.161931298668144×Albuminemia+0.000255295983138449×Albuminuria))
ii) incorporating the combined value in a software; and
iii) concluding that the subject has a high probability of maladaptive glomerular impairment when the combined value is higher than the reference value; or concluding that the subject not likely to have maladaptive glomerular impairment when the combined value is lower than the reference value.

In a further embodiment, the invention relates to a computer-implemented method for estimating the probability of maladaptive glomerular impairment in a subject comprising the following steps:
i) calculating a combined value (p) with the following formula:

p=1−1/(1+Exp((−5.69662497682848)+(0.00625296091662126*MIA+0.0243941810080423×Age−0.0360256134329782×eGFR+0.161931298668144×serum protein+0.000255295983138449×proteinuria)),
ii) incorporating the combined value in a software; and
iii) concluding that the subject has a high probability of maladaptive glomerular impairment when the combined value is higher than the reference value; or concluding that the subject not likely to have maladaptive glomerular impairment when the combined value is lower than the reference value.

In a particular embodiment, the invention relates to a computer-implemented method for discriminating primary Focal segmental glomerulosclerosis (FSGS) from maladaptive FSGS in a subject comprising the following steps:
i) obtaining a biological sample from said subject;
ii) determining mean interglomerular area (MIA),
iii) incorporating the MIA value determined at step ii) in a software; and
iv) concluding that the subject is susceptible to suffer or is suffering from primary FSGS when the MIA value is lower than the reference value; or concluding that the subject is not susceptible to suffer or is not suffering from primary FSGS when the MIA value is higher than the reference value.

In a further embodiment, the invention relates to a computer-implemented method for discriminating primary Focal segmental glomerulosclerosis (FSGS) from maladaptive FSGS in a subject comprising the following steps:
i) calculating a combined value (p) with the following formula as described above with age, estimated glomerular filtration rate (eGFR), level of proteinuria or albuminuria;
ii) incorporating the combined with age, estimated glomerular filtration rate (eGFR), and level of proteinuria in a software; and
iii) concluding that the subject is susceptible to suffer or is suffering from primary FSGS when the combined value is lower than the reference value; or concluding that the subject is not susceptible to suffer or is not suffering from primary FSGS when the combined value is higher than the reference value.

In a further embodiment, the method of the present invention is implemented by a computer executing code instructions stored on a memory.

More particularly, the invention relates to a computer-implemented method for predicting whether a subject suffering from primary or maladaptive FSGS will achieve a response to an immunosuppressive treatment comprising the following steps:
i) Obtaining a biological sample from said subject;
ii) determining mean interglomerular area (MIA)
iii) incorporating MIA value determined at step ii) in a software; and
iv) concluding that the subject will achieve a response to an immunosuppressive treatment when MIA is lower than the reference value; or concluding that the subject will not achieve a response to an immunosuppressive treatment when MIA is higher than the reference value.

In a particular embodiment, the invention relates to a computer-implemented method for predicting whether a subject suffering from primary or maladaptive FSGS will achieve a response to an immunosuppressive treatment comprising the following steps:
i) calculating a combined value (p) with the following formula as described above with age, estimated glomerular filtration rate (eGFR), level of proteinuria or albuminuria
ii) incorporating the combined value iii) in a software; and
iii) concluding that the subject will achieve a response to an immunosuppressive treatment when combined value is lower than the reference value; or concluding that the subject will not achieve a response to an immunosuppressive treatment when combined value is higher than the reference value.

In a particular embodiment, the invention relates to a computer-implemented method for predicting the probability of response to an immunosuppressive treatment in a subject suffering from primary or maladaptive FSGS comprising the following steps:
i) obtaining a biological sample from said subject;
ii) determining mean interglomerular area (MIA);
iii) incorporating MIA value determined at step ii) in a software; and
iv) concluding that the probability that the subject will respond to an immunosuppressive treatment when MIA is lower than the reference value; or concluding that the subject will likely not respond to an immunosuppressive treatment when MIA is higher than the reference value.

In a particular embodiment, the invention relates to a computer-implemented method for predicting the probability of response to an immunosuppressive treatment in a subject suffering from primary or maladaptive FSGS comprising the following steps:
i) calculating a combined value (p) with the following formula as described above with age, estimated glomerular filtration rate (eGFR), level of proteinuria or albuminuria
ii) incorporating the combined value in a software; and
iii) concluding that the probability that the subject will respond to an immunosuppressive treatment when the combined value is lower than the reference value; or concluding that the subject will likely not respond to an immunosuppressive treatment when combined value is higher than the reference value.

In the context of the invention, the analyses were performed with R software (R Development Core Team, version 1.0.44) and GraphPad PRISM® Software (GraphPad Software, San Diego, USA, version 5.02).

In a sixth aspect, the invention relates to a kit for performing the method according to the invention, wherein said kit comprises (i) means for determining MIA with a biological sample obtained from said subject, ii) means for calculating a combined value with MIA and age, estimated glomerular filtration rate (eGFR), level of proteinuria or albuminuria and (iii) instructions use.

In a further embodiment, the kit according to the invention further comprises instructions to calculate MIA value and its association with age, estimated glomerular filtration rate (eGFR), level of proteinuria and albuminuria.

Typically, the kit comprises means for measuring MIA estimated glomerular filtration rate (eGFR), level of proteinuria and/or albuminuria. Typically, the kits described above will also comprise one or more other containers, containing for example, wash reagents, and/or other reagents capable of performing the renal biopsy analysis. The kit also contains instructions suitable for incorporating MIA value and its association with age, estimated glomerular filtration rate (eGFR), level of proteinuria and albuminuria in a software.

Typically, compartmentalised kit includes any kit in which reagents are contained in separate containers, and may include small glass containers, plastic containers or strips of plastic or paper. Such containers may allow the efficient transfer of reagents from one compartment to another compartment whilst avoiding cross-contamination of the samples and reagents, and the addition of agents or solutions of each container from one compartment to another in a quantitative fashion. Such kits may also include a container which will accept the renal biopsy sample, a container which contains the antibody(s) used in the assay, containers which contain wash reagents (such as phosphate buffered saline, Tris-buffers, and like), and containers which contain the detection reagent.

The invention will be further illustrated by the following figures and examples. However, these examples and figures should not be interpreted in any way as limiting the scope of the present invention.

### FIGURES

FIG. 1: The MIA is calculated by dividing the total interglomerular area (minimum area between glomeruli, except GSG, in light grey) by the number of glomeruli (except GSG, in dark grey) minus one.

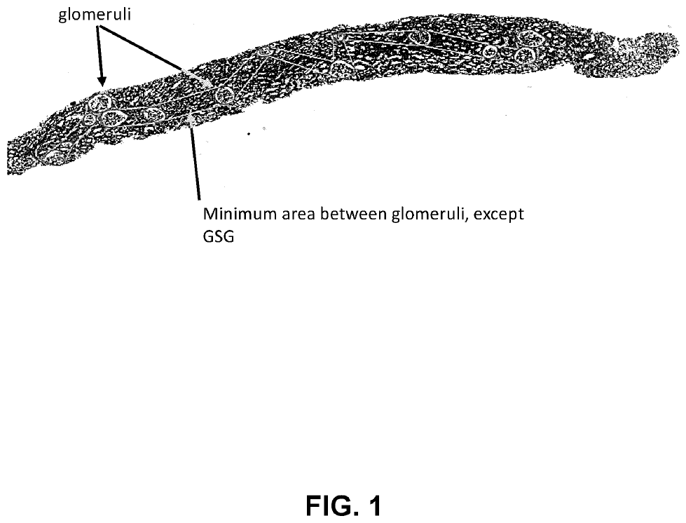

FIG. 2. Mean interglomerular area (MIA) in observation and validation cohort and ROC curves, MIA threshold with sensibility and specificity. AUROC: arca under receiver operating characteristic; CI: confidence interval; FSGS: focal segmental glomerulosclerosis; MIA: mean interglomerular area; ROC: receiver operating characteristic. Statistical analysis was carried out using GraphPad Prism 9.0.0 (GraphPad Software, San Diego, California, USA). Quantitative variables were compared using Mann-Whitney test. A value of double-sided p<0.05 was considered statistically significant.

FIG. 3: Concordance between MIA and glomerulomegaly in maladaptive FSGS (mFSGS) and primary FSGS (pFSGS) patients. In dark grey, mFSGS, in light grey, FSGS.

### EXAMPLE

### Material & Methods

The Tenon hospital Renal Human Pathology Database was searched for adult patients with an FSGS diagnosis on native renal biopsy from January 2002 through January 2020. Primary FSGS was defined as a steroid-sensitive NS (remission obtained within 4 weeks after starting steroid). Maladaptive FSGS was defined by gradually increasing proteinuria, with at least one cause of nephron number reduction (hypertension, obesity, medications, sickle cell anemia). An observational cohort with 40 patients (20 patients with primary FSGS and maladaptive FSGS, respectively) was carried out to identify renal morphometric parameters of interest. In addition, a validation cohort with 40 patients (20 patients with primary FSGS and maladaptive FSGS, respectively) was established to confirm the results matching age, estimated glomerular filtration rate (eGFR), and level of proteinuria. The inventors excluded patients with missing clinical data at the time of renal biopsy (serum albumin, proteinuria) or if renal biopsies were unavailable. The clinical data and the pathology findings (optical, immunofluorescence, and EM when available) were retrieved from the electronic medical records.

All biopsy sections were scanned (Pathscan Combi, Excilone), allowing to obtain high-definition WSI. The morphometric evaluation was performed with QuPath5, including glomeruli count, glomerular area, total biopsy area, cortical area, interglomerular area (Table 2). The renal biopsy of each patient was analyzed, blinded to the clinical data, by two nephrologists separately.

### Results

Twenty-eight patients in the observational cohort and 30 patients in the validation cohort were included. There were no statistically significant differences concerning age, gender, body mass index, baseline serum creatinine, eGFR, or hematuria (Table 1) between primary and maladaptive FSGS. However, patients with primary FSGS had lower serum albumin than maladaptive FSGSs in the observational cohort but not in the matched validation cohort. In addition, no significant difference was observed regarding the mean number of globally sclerotic glomeruli (GSG) per biopsy section.

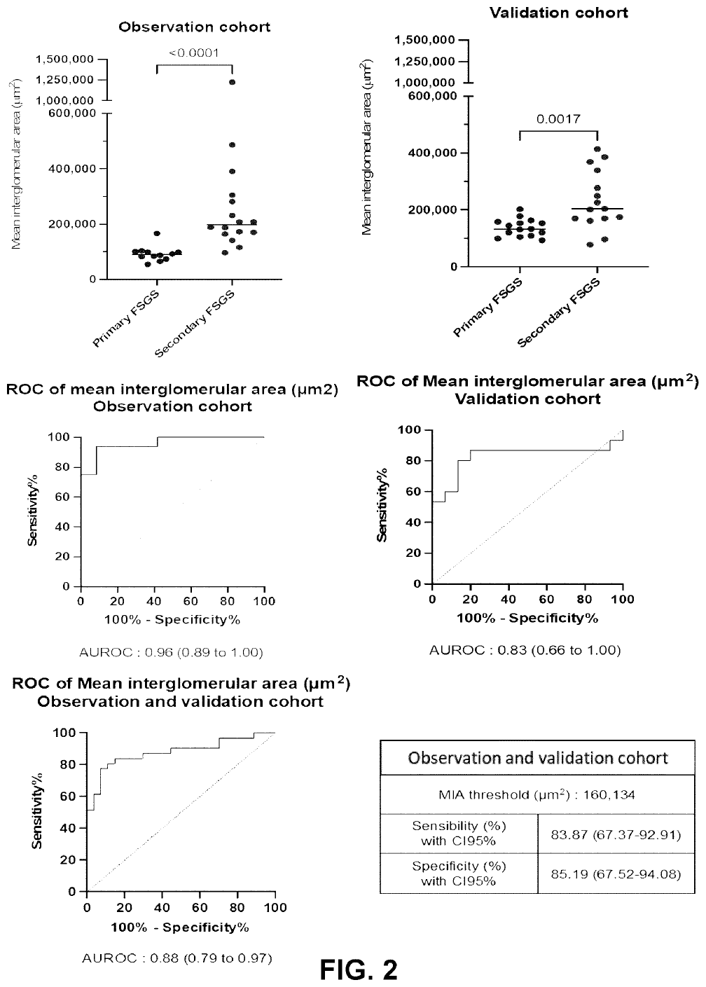

In the observational cohort, the inventors found that the mean interglomerular area (MIA, a marker of glomerular scarcity) was significantly lower in patients with primary FSGS compared to maladaptive FSGS 90 [76-100] μm² vs. 198 [165-299] μm², p<0.0001 (FIG. 2). This finding was confirmed in the validation cohort 133 [109-159] vs. 204 [170-339] μm2 (p=0.0017).

MIA predictive performance measured by area under ROC (AUROC) curve was 0.96 (95% CI 0.89-1) and 0.83 (95% CI 0.66-1) for observation and validation cohort, respectively (FIG. 2). Glomerulomegaly, a classical parameter associated with mFSGS was more frequent in patients with mFSGS, but this did not reach statistical significance (p=0.07, Table 1).

The discriminative value of MIA was confirmed in the validation cohort comparing patients matched on eGFR and proteinuria: 133 [109-159] μm2 in pFSGS vs. 204 [170-339] μm2 in mFSGS (p=0.0017, Table 1). Among other potential markers of mFSGS, serum albumin, percentage of globally sclerotic glomeruli, and interstitial fibrosis failed to discriminate between the two groups. However, glomerulomegaly was very significantly associated with mFSGS in this cohort: 13% in pFSGS vs 80% in mFSGS (p=0.0007, Table 1).

MIA predictive performance measured by area under ROC (AUROC) curve was 0.96 (95% CI [0.89-1]), 0.83 (95% CI [0.66-1]) and 0.88 (95% CI [0.79-0.97]) for the observation cohort, the validation cohort, and both, respectively (FIG. 2). The inventors determined an MIA cut-off value of 160,134 μm2 to discriminate primary FSGS from maladaptive FSGS with a sensitivity of 84% (95% CI [67-93]) and a specificity of 85% (95% CI [68-94]).

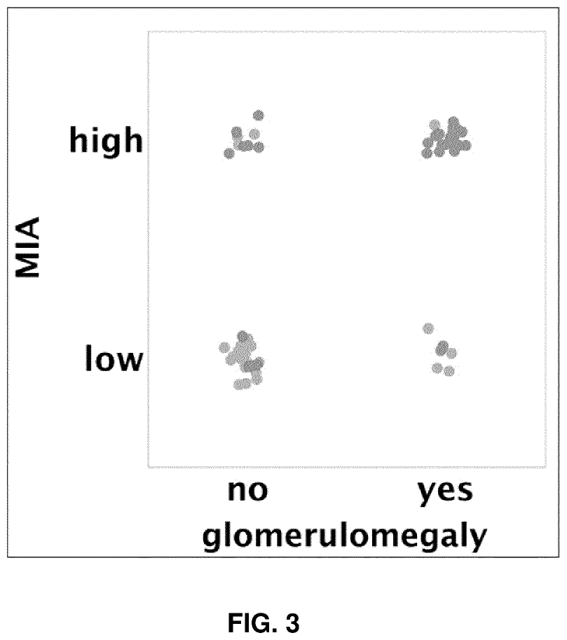

Interestingly, using both MIA and glomerulomegaly provided synergistic discrimination to identify maladaptive FSGS: out of 31 maladaptive FSGS, only 22 (71%) had glomerulomegaly, whereas 26 (84%) had a high MIA and 28 (90%) had either a high MIA or glomerulomegaly (FIG. 3).

The distinction between primary and maladaptive FSGS is challenging, resulting in unnecessary and potentially harmful immunosuppressive therapy in patients with mFSGS. Conversely, pFSGS might be considered less likely in patients with preexisting chronic conditions like chronic hypertension, which may lead to undertreatment. Therefore, the inventors propose to use MIA, a morphometric parameter easily assessable on digitalized histology slides, to predict corticosteroid sensitivity. In the present population of FSGS patients with otherwise similar clinical features, the prediction with the proposed MIA threshold is 85% accurate.

One methodological limitation of previous studies evaluating diagnostic markers of FSGS was the absence of a reliable gold standard for pFSGS. The inventors chose to bypass this issue by restricting pFSGS to a subset of patients with ascertained steroid sensitivity. This criterion is more clinically relevant than the usual dichotomy between nephrotic and non-nephrotic patients, which is only a surrogate marker. Based on studies using this surrogate marker, electronic microscopy is recommended for the classification of FSGS, as diffuse (>80%) foot process effacement is well associated with nephrotic syndrome in FSGS patients. Although valuable markers of the extent of podocyte injury, neither nephrotic syndrome nor foot process effacement can predict steroid sensitivity in FSGS. Furthermore, electronic microscopy is not universally available.

MIA is a geometrical value depending on both the interglomerular distance and glomerular diameter. Thus, MIA is increased when glomeruli are large and interspaced. Increased glomerular diameter, i.e. glomerulomegaly, is a classical feature of mFSGS, especially in obesity-related FSGS. However, in the present discovery and validation cohorts, the sensitivity of this parameter for mFSGS was only 63% and 80% respectively. MIA depends on glomerular dispersion in addition to glomerular size and surpasses glomerulomegaly to predict mFSGS (sensitivity 81% and 87% for the discovery and validation cohorts, respectively).

TABLE 1
Baseline demographics, clinical and biological data from the observation cohort and validation cohort

| Observation cohort | | | Validation Cohort |
|---|---|---|---|
| Primary | Maladaptive | | Primary |
| FSGS | FSGS | p- | FSGS |
| n = 12 | N = 16 | value | n = 15 |

Baseline demographics

| Female, N. (%) | 4 | (33%) | 9 | (56%) | 0.28 | 8 | (53%) |
|---|---|---|---|---|---|---|---|
| Age (years) | 44 ± 22 | | 55 ± 16 | | 0.16 | 37 | [31-62] |
| Caucasian, N. (%) | 7 | (58%) | 12 | (75%) | 0.43 | 10 | (67%) |

| | | | | |
|---|---|---|---|---|
| BMI (kg/m²) | 25.9 ± 5.6 | 26.9 ± 6.3 | 0.71 | 24.8 ± 4 |

| | | | | | | | |
|---|---|---|---|---|---|---|---|
| Active cancer, N. (%) | 0 | (0%) | 3 | (19%) | 0.24 | 0 | (0%) |
| Diabetes, N. (%) | 3 | (25%) | 3 | (19%) | >0.99 | 3 | (20%) |
| Hypertension, N. (%) | 7 | (58%) | 12 | (75%) | 0.43 | 7 | (47%) |
| HIV infection, N. (%) | 0 | (0%) | 0 | (0%) | >0.99 | 0 | (0%) |
| HBV infection, N. (%) | 0 | (0%) | 0 | (0%) | >0.99 | 2 | (13%) |
| HCV infection, N. (%) | 0 | (0%) | 1 | (6%) | >0.99 | 0 | (0%) |
| Tobacco use, N. (%) | 5 | (42%) | 5 | (31%) | 0.70 | 3 | (20%) |

Clinical and Biological data at the time of renal biobsy

| | | | | | |
|---|---|---|---|---|---|
| Systolic blood | 140 ± 20 | 137 ± 14 | 0.64 | 133 | [122-154] |

pressure (mmHg)

| | | | | | |
|---|---|---|---|---|---|
| Diastolic blood | 85 ± 13 | 80 ± 13 | 0.31 | 82 | [71-88] |

pressure (mmHg)

| | | | | | | | |
|---|---|---|---|---|---|---|---|
| Creatinine (μmol/L) | 186 | [95-337] | 141 | [106-219] | 0.38 | 107 | [87-135] |

| | | | | |
|---|---|---|---|---|
| eGFR | 42 ± 34 | 39 ± 16 | 0.70 | 56 ± 22 |
| (mL/min/1.73 m², | | | | |
| CKD-Epi) | | | | |
| Serum albumin (g/L) | 16.1 ± 6.6 | 36 ± 6.8 | <0.0001 | 25.7 ± 10.5 |

| | | | | | | | |
|---|---|---|---|---|---|---|---|
| Urinary | 790 | [483-1345] | 420 | [188-675] | 0.07 | 970 | [230-1174] |

protein/creatinine
ratio (mg/mmol)

| | | | | | | | |
|---|---|---|---|---|---|---|---|
| Urinary | 559 | [364-686] | 270 | [124-460] | 0.09 | 550 | [323-896] |

albumin/creatinine
ratio (mg/mmol)

| | | | | | | | |
|---|---|---|---|---|---|---|---|
| Hematuria, N. (%) | 2 | (17%) | 2 | (12%) | >0.99 | 2 | (13%) |
| Leukocyturia, N. (%) | 3 | (25%) | 2 | (12%) | >0.99 | 4 | (27%) |

| Histological and morphometric parameters | | | | |
|---|---|---|---|---|
| FSGS variant | 2/3/10/6 | 3/1/12/9 | 0.44 | 2/6/00/7 |
| Collapsing/tip/cellular | | | | |
| perihilar/NOS | | | | |

| | | | | | | | |
|---|---|---|---|---|---|---|---|
| Glomeruli per biopsy | 16 | [13.5-21.8] | 5.25 | [3-9.9] | 0.0001 | 7.4 | [6.7-14.5] |

section (excluding
GSG), N. (%)

| | | | | | | | |
|---|---|---|---|---|---|---|---|
| GSG per biopsy | 0 | [0-1] | 0 | [0-3] | 0.49 | 0 | [0-0.7] |

section, N. (%)

| | | | | | | | |
|---|---|---|---|---|---|---|---|
| Interstitial fibrosis (%) | 5 | [0-30] | 30 | [17.5-42.5] | 0.04 | 10 | [2.5-35] |
| Glomerulomegaly (%) | 3 | (25%) | 10 | (63%) | 0.07 | 2 | (13%) |
| MIA >160, 134 μm² | 1 | (8%) | 13 | (81%) | 0.0003 | 3 | (20%) |
| MIA (μm²) | 89,807 | [75,655-99,745] | 198,192 | [164,986-298,689] | <0.0001 | 133,163 | [109,450-158,722] |

| | Validation Cohort | | |
|---|---|---|---|
| | Maladaptive | | |
| | FSGS | | p- |
| | n = 15 | | value |
| Baseline demographics | | | |
| Female, N. (%) | 7 | (47%) | >0.99 |
| Age (years) | 56 | [35-67] | 0.11 |
| Caucasian, N. (%) | 7 | (47%) | 0.46 |
| BMI (kg/m²) | 27 ± 5 | | 0.91 |
| Active cancer, N. (%) | 1 | (7%) | >0.99 |
| Diabetes, N. (%) | 5 | (33%) | 0.68 |
| Hypertension, N. (%) | 9 | (60%) | 0.72 |
| HIV infection, N. (%) | 0 | (0%) | >0.99 |
| HBV infection, N. (%) | 0 | (0%) | 0.48 |
| HCV infection, N. (%) | 0 | (0%) | >0.99 |
| Tobacco use, N. (%) | 7 | (47%) | 0.25 |
| Clinical and Biological data at the time of renal biobsy | | | |
| Systolic blood | 140 | [122-155] | 0.77 |
| pressure (mmHg) | | | |
| Diastolic blood | 78 | [71-89] | 0.99 |
| pressure (mmHg) | | | |
| Creatinine (μmol/L) | 92 | [82-166] | 0.41 |
| eGFR | 63 ± 28 | | 0.50 |
| (mL/min/1.73 m², | | | |
| CKD-Epi) | | | |
| Serum albumin (g/L) | 31.9 ± 11.1 | | 0.18 |
| Urinary | 305 | [115-954] | 0.27 |
| protein/creatinine | | | |
| ratio (mg/mmol) | | | |
| Urinary | 209 | [88-797] | 0.07 |
| albumin/creatinine | | | |
| ratio (mg/mmol) | | | |
| Hematuria, N. (%) | 0 | (0%) | 0.48 |
| Leukocyturia, N. (%) | 1 | (7%) | 0.33 |
| Histological and morphometric parameters | | | |
| FSGS variant | 0/0/01/14 | | 0.0022 |
| Collapsing/tip/cellular | | | |
| perihilar/NOS | | | |
| Glomeruli per biopsy | 7 | [5-9] | 0.13 |
| section (excluding | | | |
| GSG), N. (%) | | | |
| GSG per biopsy | 0.5 | [0-1.7] | 0.24 |
| section, N. (%) | | | |
| Interstitial fibrosis (%) | 15 | [5-45] | 0.52 |
| Glomerulomegaly (%) | 12 | (80%) | 0.0007 |
| MIA >160, 134 μm² | 13 | (87%) | 0.0007 |
| MIA (μm²) | 204,390 | [169,753-339,350] | 0.0017 |

BMI: body mass index; eGFR: estimated glomerular filtration rate; FSGS: focal segmental glomerulosclerosis; GSG: globally sclerotic glomeruli; HBV: hepatitis B virus; HCV: hepatitis C virus; HIV: human immunodeficiency virus; MIA: mean interglomerular area; Hematuria was defined as >104 red blood cells/ml and leukocyturia as >10⁴ leukocytes/ml. Statistical analysis was carried out using GraphPad Prism 9.0.0 (GraphPad Software, San Diego, California, USA).
Continuous variables are reported as the mean ± standard deviation or median [interquartile range] if the variable did not correspond to a normal distribution and categorical variables are reported as numbers (percentages). Quantitative variables were compared using a t-test (normal distribution) or Mann-Whitney test and qualitative variables were compared using Fisher's exact test. A value of double-sided p < 0.05 was considered statistically significant.

TABLE 2
The morphometric evaluation was performed with QuPath5, including glomeruli count,
glomerular area, total biopsy area, cortical area, interglomerular area.

| | | | Centroid X | Centroid Y | Area | Perimeter |
|---|---|---|---|---|---|---|
| Image | Name | ROI | μm | μm | μm\hat{ }2 | μm |
| Primary FSGS XXX | Minimal | polygon | 1894.156 | 631.048 | 1052838.556 | 8877.792 |
| | cortical area | | | | | |
| Primary FSGS XXX | Glomeruli 1 | polygon | 1026.96 | 917.488 | 31179.94 | 446.424 |
| Primary FSGS XXX | Glomeruli 2 | polygon | 2247.564 | 301.7696 | 6911.74 | 216.0532 |
| Primary FSGS XXX | Glomeruli 3 | polygon | 3244.34 | 416.6536 | 19325.46 | 348.4888 |
| Primary FSGS XXX | Glomeruli 4 | polygon | 600.644 | 954.536 | 14431.56 | 298.232 |
| Primary FSGS XXX | Glomeruli 5 | polygon | 3512.036 | 316.7472 | 44304.04 | 535.216 |
| Primary FSGS XXX | Glomeruli 6 | polygon | 2787.004 | 308.902 | 31647.88 | 456.808 |
| Primary FSGS XXX | Glomeruli 7 | polygon | 2262.964 | 559.196 | 42291.04 | 515.856 |
| Primary FSGS XXX | Glomeruli 8 | polygon | 369.1204 | 1258.18 | 28847.94 | 435.3888 |
| Primary FSGS XXX | Glomeruli 9 | polygon | 1492.392 | 762.08 | 33525.58 | 464.4948 |
| Primary FSGS XXX | Glomeruli 10 | polygon | 450.648 | 1150.468 | 6480.54 | 214.9708 |
| Primary FSGS XXX | Glomeruli 11 | polygon | 3478.2 | 452.276 | 37122.8 | 474.892 |
| Primary FSGS XXX | Glomeruli 12 | polygon | 757.064 | 943.712 | 42705.52 | 529.144 |
| Primary FSGS XXX | Glomeruli 13 | polygon | 670.56 | 826.144 | 41421.82 | 512.38 |
| Primary FSGS XXX | Glomeruli 14 | polygon | 1760.352 | 419.1924 | 14807.1 | 314.1644 |

Total number of glomeruli (except GSC) = 14
Minimal cortical area = 1052838.56 μm\hat{ }2
Mean interglomerular area (MIA) = (minimal cortical area/(total number of glomeruli except GSC − 1)) = 80987.58

### REFERENCES

1. De Vriese, A. S., Sethi, S., Nath, K. A., Glassock, R. J. & Fervenza, F. C. Differentiating Primary, Genetic, and Secondary FSGS in Adults: A Clinicopathologic Approach. J. Am. Soc. Nephrol. 29, 759-774 (2018).
2. Korbet, S. M. Treatment of Primary FSGS in Adults. J. Am. Soc. Nephrol. 23, 1769-1776 (2012).
3. Sethi, S., Glassock, R. J. & Fervenza, F. C. Focal segmental glomerulosclerosis: towards a better understanding for the practicing nephrologist. Nephrol. Dial. Transplant. 30, 375-384 (2015).
4. Sethi, S., Zand, L., Nasr, S. H., Glassock, R. J. & Fervenza, F. C. Focal and segmental glomerulosclerosis: clinical and kidney biopsy correlations. Clin. Kidney J. 7, 531-537 (2014).
5. Bankhead, P. et al. QuPath: Open source software for digital pathology image analysis. Sci. Rep. 7, 16878 (2017).

1-14. (canceled)

15. An in vitro method for discriminating primary Focal segmental glomerulosclerosis (FSGS) from maladaptive FSGS in a subject comprising:
determining a mean interglomerular area (MIA) value in a biological sample obtained from said subject,
wherein said subject is susceptible to suffer or is suffering from primary FSGS when the MIA value is lower than a reference value; or the subject is susceptible to suffer or is suffering from maladaptive FSGS when the MIA value is higher than the reference value.

16. The method according to claim 15 wherein the MIA value is calculated with the following formula: MIA=(minimal cortical area/total number of glomeruli except segmental glomerulosclerosis (GSG)−1)).

17. The method according to claim 15 further comprising determining a proteinuria level in said biological sample, wherein the subject is susceptible to suffer or is suffering from primary FSGS when the proteinuria level is higher than a reference value, or the subject is susceptible to suffer or is suffering from maladaptive FSGS when proteinuria level is lower than the reference value.

18. The method according to claim 15 further comprising determining a level of serum protein in said biological sample, wherein the subject is susceptible to suffer or is suffering from primary FSGS when the level of serum protein is lower than a reference value, or the subject is susceptible to suffer or is suffering from maladaptive FSGS when the level of serum protein is higher than the reference value.

19. The method according to claim 15 further comprising determining a level of serum albumin in the biological sample, wherein the subject is susceptible to suffer or is suffering from primary FSGS when the level of serum albumin is lower than a reference value, or the subject is susceptible to suffer or is suffering from maladaptive FSGS when the serum albumin level is higher than the reference value.

20. The method according to claim 15 further comprising determining an estimated Glomerular Filtration Rate (eGFR) in the biological sample, wherein the subject is susceptible to suffer or is suffering from primary FSGS when the eGFR is lower than a reference value, or the subject is susceptible to suffer or is suffering from maladaptive FSGS when the eGFR is higher than the reference value.

21. The method according to claim 15 wherein the biological sample is a renal biopsy sample.

22. The method according to claim 15 wherein a subject suffering from FSGS is predicted to respond to an immunosuppressive treatment when the MIA value is lower than the reference value; or not to respond to an immunosuppressive treatment when the MIA value is higher than the reference value.

23. The method according to claim 22 wherein the MIA value is calculated with the following formula: MIA=(minimal cortical area/total number of glomeruli except segmental glomerulosclerosis (GSG)−1)).

23. The method according to claim 22 wherein the biological sample is a renal biopsy sample.

24. A method for treating primary Focal segmental glomerulosclerosis in a subject in need thereof, comprising administering to the subject a therapeutically efficient amount of an immunosuppressive drug, wherein the subject is identified as susceptible to suffer or is suffering from primary FSGS using the method according to claim 1.

25. The method of claim 24 wherein the immunosuppressive drug is selected from the group consisting of: azathioprine, tacrolimus, a rapamycin derivative, mycophenolic acid, a corticosteroid, and cyclosporin.

26. The method of claim 25 wherein the immunosuppressive drug is a corticosteroid selected from the group consisting of: cortisone, cortisol, hydrocortisone (11β,17-dihydroxy, 21-(phosphonooxy)-pregn-4-ene, 3,20-dione disodium), dihydroxycortisone, dexamethasone (21-(acetyloxy)-9-fluoro-1β,17-dihydroxy-16α-m-ethylpregna-1,4-diene-3,20-dione), and a highly derivatized steroid drug.

27. The method of claim 26 wherein the highly derivatized steroid drug is beconase (9-chloro-11-β, 17,21, trihydroxy-16β-methylpregna-1,4 diene-3,20-dione 17,21-dipropionate).

28. A computer-implemented method for performing a method for discriminating primary Focal segmental glomerulosclerosis (FSGS) from maladaptive FSGS in a subject comprising:
determining a mean interglomerular area (MIA) value in a biological sample obtained from the subject,
wherein the subject is susceptible to suffer or is suffering from primary FSGS when the MIA value is lower than a reference value; or the subject is susceptible to suffer or is suffering from maladaptive FSGS when the MIA value is higher than the reference value.

29. The computer-implemented method according to claim 28 comprising the following steps:
i) incorporating in a software the MIA value determined in the biological sample obtained from the subject; and
ii) concluding that the subject is susceptible to suffer or is suffering from primary FSGS when the MIA value is lower than the reference value; or the subject is susceptible to suffer or is suffering from maladaptive FSGS when the MIA value is higher than the reference value.

30. The computer-implemented method according to claim 28 comprising the following steps:
i) incorporating in a software a set of parameters comprising the MIA value determined in the biological sample obtained from the subject, together with at least one parameter selected from the group consisting of: an estimated glomerular filtration rate (eGFR), a proteinuria level and a serum protein level in said subject,
ii) calculating a probability of maladaptive FSGS (p) from said set of parameters, and
iii) concluding that said subject is susceptible to suffer or is suffering from primary FSGS when the probability is lower than a reference value; or the subject is susceptible to suffer or is suffering from maladaptive FSGS when said probability is higher than the reference value.

31. The computer-implemented method according to claim 30 wherein said serum protein level is a serum albumin level.
